(19) United States
(12) Patent Application Publication
(10) Pub. No.: US 2004/0186095 A1
(43) Pub. Date: Sep. 23, 2004

(54) Highly purified and crystalline form of harringtonines their process of preparation by purification of crude alkaloids from natural synthetic or semi-synthetic sources allowing their use for blending in pharmaceutical composition particularly useful for treatment of cancer in using oral mode of administration

(76) Inventors: Jean-Pierre Robin, Eguilles (FR); Julie Blanchard, Rouillon (FR); Robert Dhal, Pruille Le Chetif (FR); Jean-Pierre Marie, Sevres (FR); Nina Radosevic, Trange (FR)

Correspondence Address: BURNS DOANE SWECKER & MATHIS L L P, POST OFFICE BOX 1404, ALEXANDRIA, VA, 22313-1404, US

(21) Appl. No.: 10/472,299

(22) PCT Filed: Mar. 21, 2003
(86) PCT No.: PCT/IB02/02054

(51) Int. Cl. A61K 31/55; C07D 491/04

(52) U.S. Cl. 514/214.03; 540/576

(57) ABSTRACT

The invention concerns a natural, synthetic or semi-synthetic harringtonines including their tautomeric forms and their salts of the following formula: wherein n=2 (i.e. harringtonine) or n=3(i.e. homoharringtonine), in which the total content of impurities, possibly including enantiomeric forms, is lower than 1%, and/or the content of the major impurity is lower than 0.9%, and/or the chromatographic assay exhibits a harringtonines content higher than 97.5%.

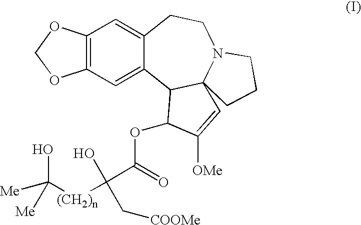

The present invention concerns highly purified and crystalline form of harringtonines, definite by their solid state analysis patterns, their process of preparation by purification of crude alkaloids from natural, synthetic or semi-synthetic sources, allowing their use as drug substance for blending alone or in combination in pharmaceutical composition particularly useful for treatment of cancer in using oral mode of administration.

Harringtonines (i.e. harringtonine=HA and homoharringtonine=HHT) are particular cephalotaxine esters, alkaloids isolated from rare and endangered conifers belonging to the Cephalotaxus genus. Cephalotaxine and its natural ester are gathered under the generic term of cephalotaxane.

### Definitions (see Scheme 1)

Cephalotaxanes are particular alkaloids today only extracted from the Cephalotaxaceae family which exhibiting the structural formula 1. Several substituants may be encountered on this core structure: hydroxyl, ether, acyloxy etc. The eventual presence of some additional double bound or intramolecular bridge achieve to definite cephalotaxanes. Cephalotaxines 2 and harringtonines 3, are examples of cephalotaxanes. Several dozen of cephalotaxanes have been isolated from various Cephalotaxus species.

Cephalotaxines 2 are cephalotaxanes without acyloxy side-chain. Cephalotaxine 2a and drupacine 2b are example of cephalotaxines.

Harringtonines 3 are particular cephalotaxanes formed by attachment of a branched hydroxyacyloxy side-chain at the 3-position of various cephalotaxines moieties. Harringtonines are natural esters of cephalotaxines exhibiting generally a strong cytotoxic activity. However the lost only one atom of this minimal structure lead to a dramatic lost of activity (see below). Some example of harringtonines are harringtonine 3a, homoharringtonine 3b, drupangtonine 3c, anhydroharringtonine 3d and neoharringtonine 3e.

Two harringtonines are very promising drugs in the treatment of certain leukemia such as Chronic Myelogenous Leukemia (CML). Compassionate use in CML patients resistant or not eligible to all existing therapies is ongoing in frace and several phase II and III clinical trials are ongoing in France and in the U.S.

Drug agencies, such as the U.S. Food and Drug Administration, require a high level of purity including enantiomeric, before approval new agents, particularly when these agents are islolated from natural sources. For example, 0.1% an impurity must be qualified and toxicology studies must be performed. New drugs not enantiomerically pure or as racemic mixture are no longer approved by the FDA. In addition, due to large variation of related impurities profil under environmental conditions, drug agencies are particularly suspicious versus drug substances prepared from direct extraction of organisms.

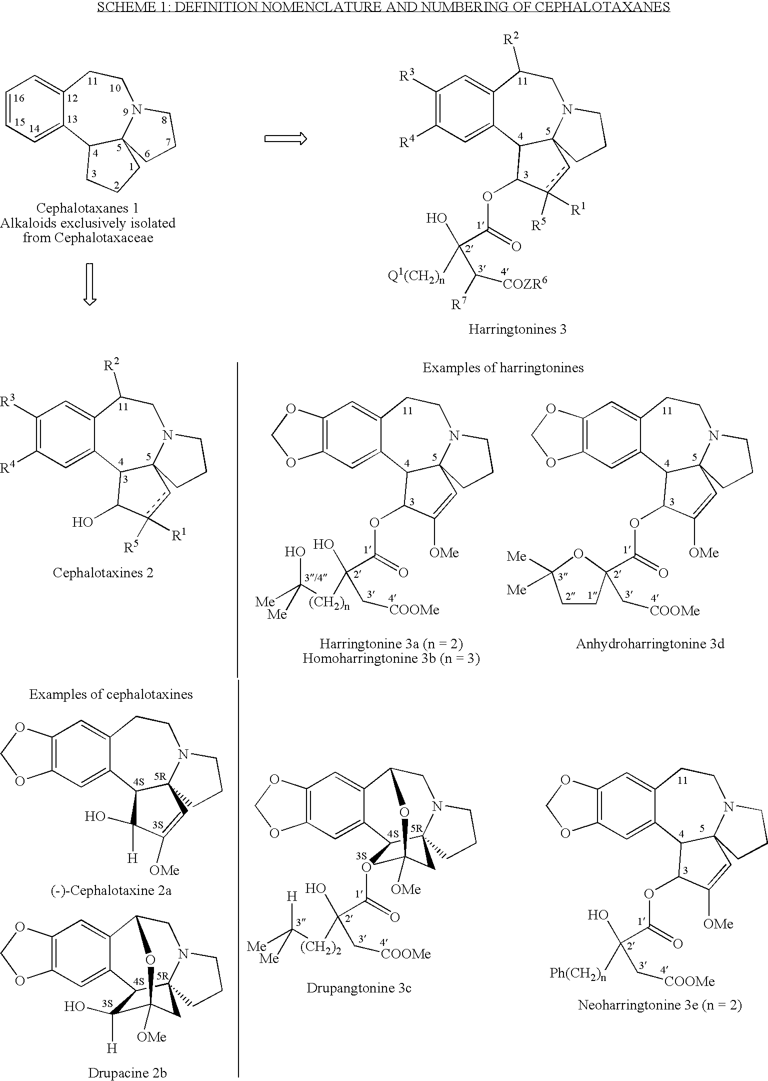

Homoharringtonine and harringtonine are present in Cephalotaxus extract as a complex mixture of several dozens of alkaloids (see scheme 1). For example, HA and HHT were firstly used as a mixture for therapy of cancer and leukemia in China. In the U.S., the level of compliance of drug substances to a given quality required by the FDA is increasing during the process of development of an investigational drug (i.e. from early phase I clinical trial to phase III). In addition, FDA requires that the profile of impurities in term of related compounds shall be reproducible from batch to batch during the marketing phase, that is very difficult when the drug substance is prepared from natural source. An homoharringtonine prepared from natural source was developed by the U.S., the National Cancer Institute and used in its early clinical trials. In despite of a final purification which use a crystallization in ethyl acetate, the final drug substance contains three major impurities including two natural congeners and ethyl analog of HHT which is an artifact of purification resulting from transesterification with the solvent of purification.[He et al., Journ. of Pharm. Biomed. Analysis, 22, pp541-534 (2000)] The following table, reproduced from reference [He et al., 2000] exemplified the quality of this HHT, which is fact the best quality yielded in using the method of purification the state of art.

TABLE I
Quality Of Various National Cancer Institute (NCI)
Clinical Batches Of Homoharringtonine Described
In Literature [1] Compared to Semi-Synthetic
Batches

| | | | | % Total of | % |
|---|---|---|---|---|---|
| | % Related | % Related | % Related | Rel. | HPLC |
| Batch Identification | Comp. A | Comp. b | Comp. c | Comp. | purity |
| NCI Batch #800528 | 0.1 | 1.3 | 0 | 1.4 | 98.6 |
| NCI Batch #871203 | 0 | 1.0 | 0 | 1.0 | 99.0 |
| NCI Batch #921115 | 0.3 | 3.0 | 0.8 | 4.1 | 95.9 |
| NCI Batch #960625 | 0 | 1.8 | 0.3 | 2.1 | 97.9 |
| NCI Batch #800722 | 0.1 | 0.9 | 0.2 | 12 | 98.8 |
| NCI Batch #KS-22-130-2 | 0 | 1.5 | 0.9 | 2.4 | 97.6 |
| Average of National Cancer | 0.1 | 1.6 | 0.4 | 2.0 | 98.0 |
| Institute batches | | | | | |
| Average of Oncopharm's | 0.00 | 0.00 | 0.00 | <0.05 | >99.95 |
| batches | | | | | |
| Rate Impurities NCI batches/ | | | | >40 | |
| Impurities Oncopham's Batches | | | | | |

[1] Stability indicating LC assay of and impurity indentification in homoharringtonine samples, He et al., Journ. of Pharm. Biomed. Analysis, 22, pp541-534 (2000).

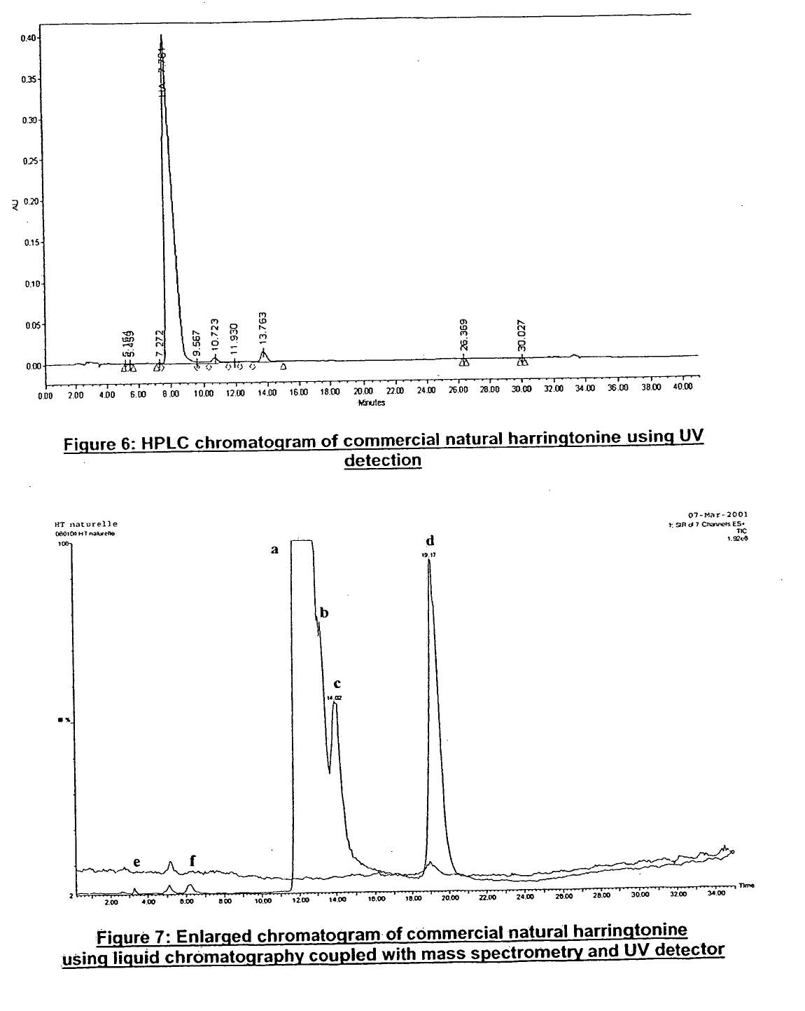

FIGS. 6, 7, 10, 12 show a chromatographic profiles of harringtonines coming from various sources.

Phase III clinical trial with an HHT drug substance exhibiting a non-reliable impurity profile.[He et al., 2000] The NCI got finally an HHT suitable for use in phase III clinical trial but, despite of its effort, the product they use contains a non-removable impurities of the which contain is higher than 1%.[He et al., 2000]. In addition to the process described for the purification of the NCI production.[He et al., 2000]

Our recent semi-synthesis of harringtonines, including harringtonine and homoharringtonine, by attachment of entirely prior formed acyl side-chains to cephalotaxine moieties, changed dramatically this situation: chromatographic purity of the final drug substance is consistently higher then 99.8% versus 98.5% for the above cited NCI products (the purest ever previously described) versus 95%-97% for Chinese products, corresponding to 0.2%, 1.5 and 3-5% of impurities (see FIGS. 6, 7 and 12). In addition, since cephalotaxine, as precursor of semi-synthetic HA and HHT, is abundant in renewable part of the tree, this semi-synthetic process overcome the serious environmental concern induce by the destroying of a rare and endanger plant.

A well definite crystalline form of a drug substance is a very important condition, to have reliable solid final form of drugs useful for example for oral administration.

Although HHT and HA would be very promising drugs for the treatment of patients with CML, the current mode of administration by continuous intravenous central infusion (CIVI) is a strong handicap for the administration of this therapy during several years. In addition, while extra-hematologic toxicity of HHT/HA is very mild, the occurrence of infection due to catheter is the main toxicity of this regimen. The use of an oral form of these drugs could be change completely this situation and would extend widely the market of this product.

The present invention provides natural, synthetic or semi-synthetic harringtonines including their tautomeric forms and their salts of the following formula:

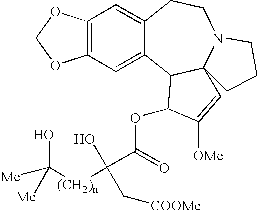

wherein n=2 (i.e. harringtonine) or n=3 (i.e. homoharringtonine), in which:

the total content of impurities, possibly including enantiomeric forms, is lower than 1%, and/or

the content of the major impurity is lower than 0.9%, and/or

the chromatographic assay exhibits a harringtonines content higher than 97.5%.

A preferred embodiment of the invention provides a natural, synthetic or semi-synthetic homoharringtonine including its tautomeric forms and its salts in which:

the total content of impurities, possibly including enantiomeric forms, is lower than 1%, and/or

the content of the major impurity is lower than 0.9%, and/or

the chromatographic assay exhibits a homoharringtonines content higher than 97.5%.

A further preferred embodiment of the invention provides a natural, synthetic or semi-synthetic harringtonine including its tautomeric forms and its salts in which:

the total content of impurities, possibly including enantiomeric forms, is lower than 1%, and/or

the content of the major impurity is lower than 0.9%, and/or

the chromatographic assay exhibits a harringtonine content higher than 97.5%.

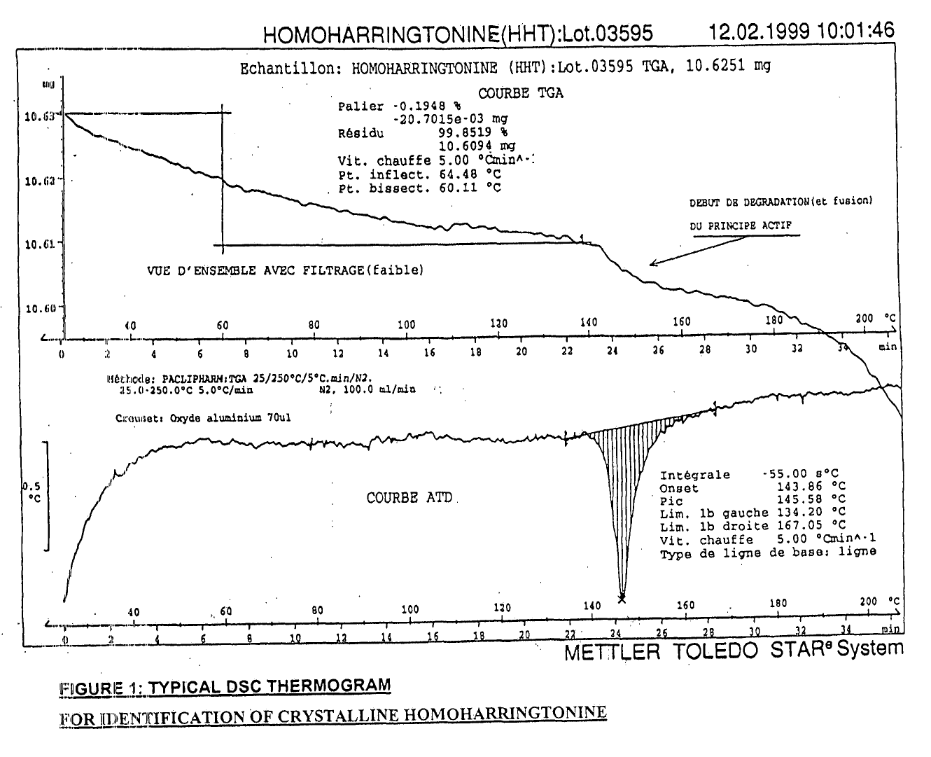

A further preferred aspect of the invention is a crystalline natural, synthetic or semi-synthetic homoharringtonine having substantially the same DSC curve as set out in FIG. 1

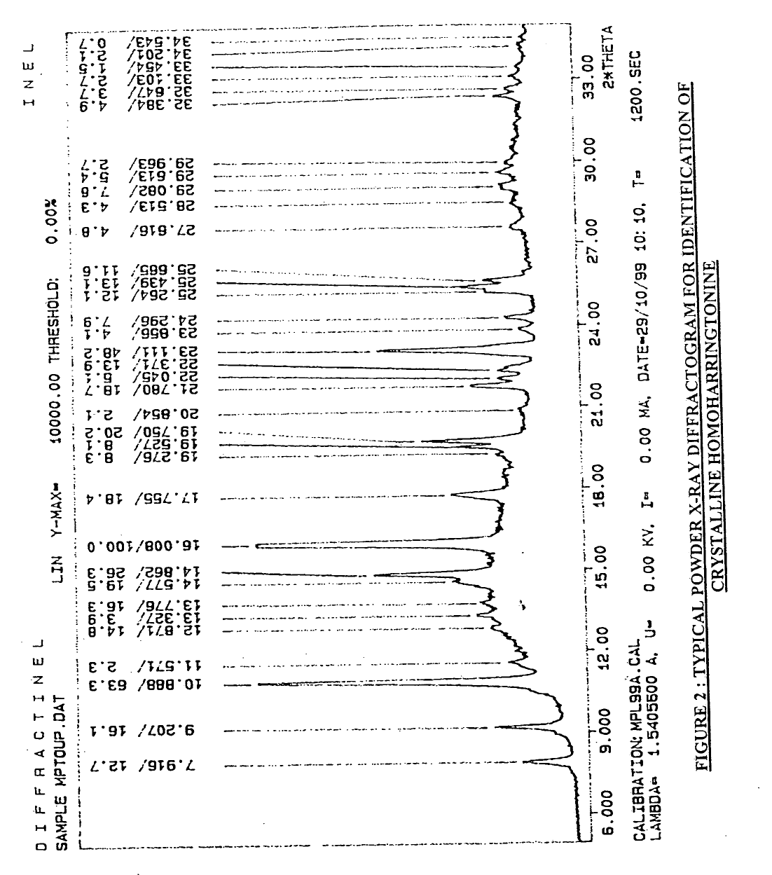

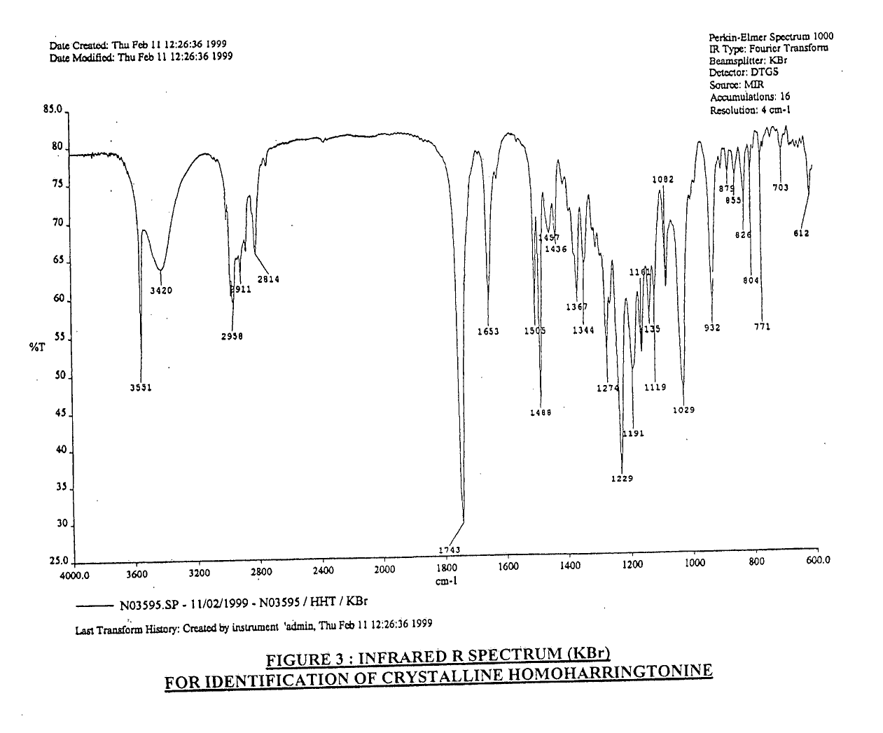

Yet, a further embodiment of the invention provides a crystalline natural, synthetic or semi-synthetic homoharringtonine having substantially the same X-ray diffractogram as set out in FIG. 2, and substantially the same IR spectrum, in KBr as set out in FIG. 3. Yet, another embodiment of the invention provides a crystalline natural, synthetic or semi-synthetic homoharringtonine having substantially the same DSC curve as set out in FIG. 1, and substantially the same X-ray diffractogram as set out in FIG. 2, and substantially the same IR spectrum, in KBr as set out in FIG. 3.

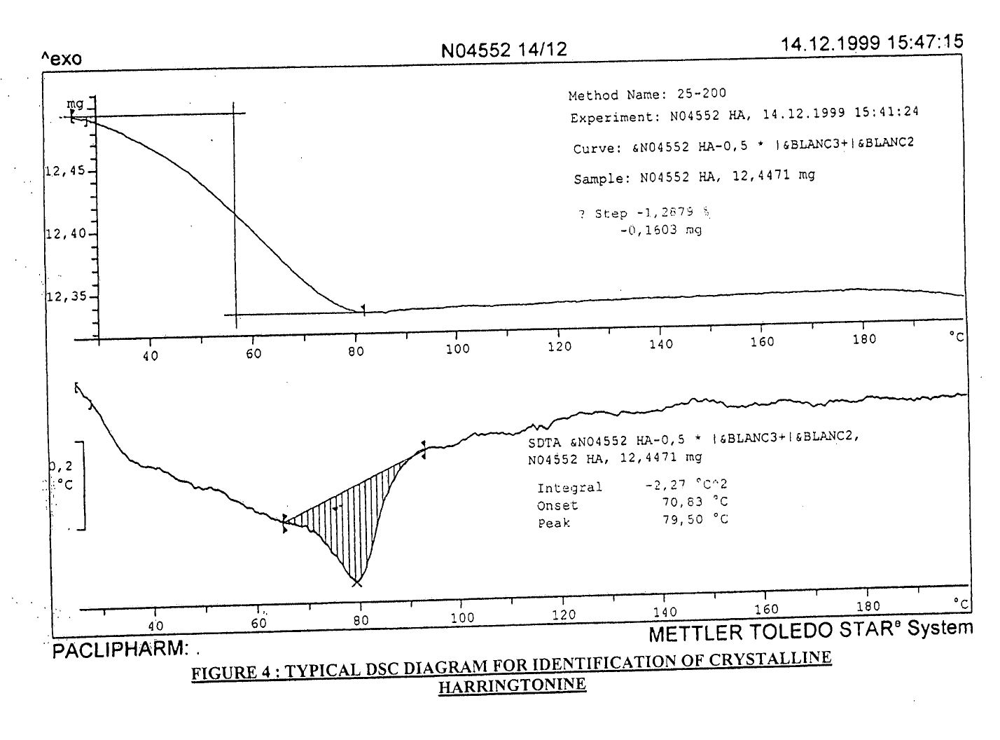

Yet, another preferred embodiment of the invention provides a crystalline natural, synthetic or semi-synthetic harringtonine having substantially the same DSC curve as set out in FIG. 4.

Yet, a preferred aspect of this invention provides a pharmaceutical composition comprising an effective antitumor amount of a natural, synthetic or semi-synthetic homoharringtonine having substantially the same X-ray diffractogram as set out in FIG. 2, and substantially the same IR spectrum, in KBr as set out in FIG. 3, and substantially the same DSC curve as set out in FIG. 1, together with one or more pharmaceutically acceptable inactive components such as carriers, excipients, adjuvants or diluents.

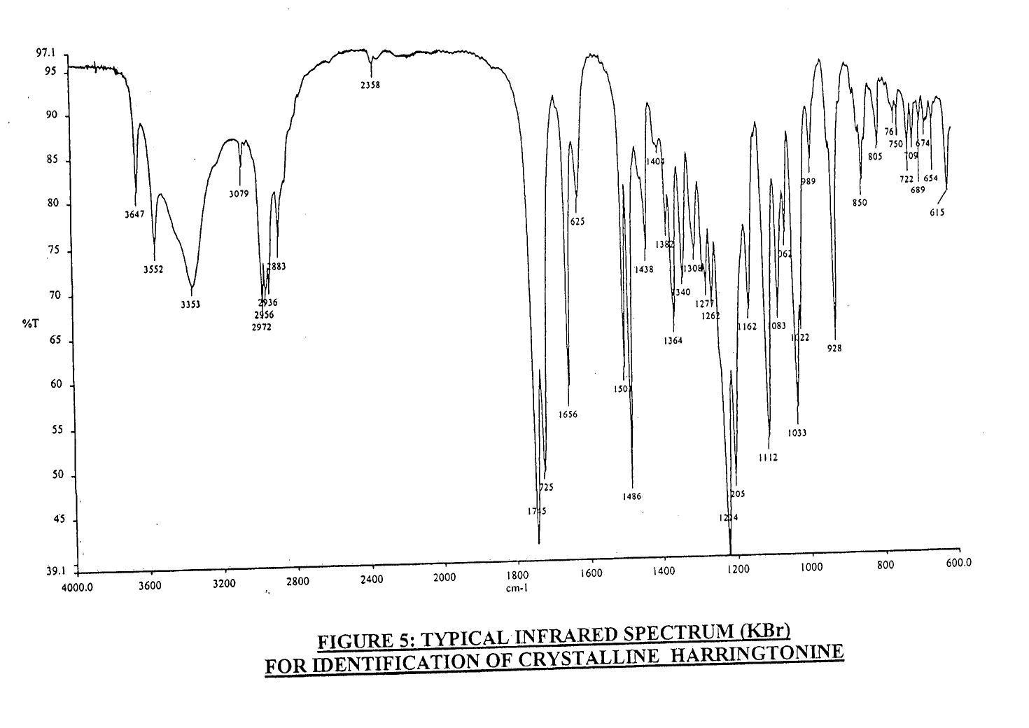

Another aspect of the invention provides a pharmaceutical composition comprising an effective antitumor amount of a natural, synthetic or semi-synthetic harringtonine having substantially the same IR spectrum, in KBr as set out in FIG. 5, and substantially the same DSC curve as set out in FIG. 4, together with one or more pharmaceutically acceptable inactive components such as carriers, excipients, adjuvants or diluents.

Another preferred aspect of the invention provide a process of purification of natural, synthetic or semi-synthetic crude harringtonines for the preparation of pure harringtonines exhibiting the above included features including for eventual enantiomeric enrichment, and comprising the successive steps:

(i) at least one chromatographic purification, preferably in reverse phase in aqueous mobile phase such as a lower alkanol or tetrahydrofurane or acetonitrile, purified water, and an acidic buffer, preferably based on phosphoric acid and is salt. Stationary phase may be any standard chemically bound phase preferably an alkylsilane or an alkylnitrile, bounded on an inert core, preferably silicagel;

(ii) at least one crystallization in water or aqueous solvent containing an organic solvent, preferably a lower C₁₋₄ alkanol,

The progression of the process of purification is monitored by HPLC analyses and several termal analysis at the solid state. The progression of enantiomeric purity is monitored by optical rotation checking of the dried solid form.

A preferred embodiment provides a new method of monitoring of enantiomeric purity of cephalotaxanes using an HPLC with a chiral stationary phase preferably based upon beta-cyclodextrine

Another preferred embodiment of the invention is the above process of purification in which the lower C₁₋₄ alkanol is methanol and the cephalotaxane is harringtonine

A further preferred aspect of the invention is the above process of purification in which the lower C₁₋₄ alkanol is methanol and the cephalotaxane is homoharringtonine

This invention include also a pharmaceutical composition which comprises an antitumor effective amount of at least one above described harringtonine or homoharringtonine with one or more pharmaceutically acceptable carriers, excipients or diluents therefore, including the process for preparing the said solid pharmaceutical composition such as, for examples, tablet, capsule, implant or suppository.

Another aspect of the invention is the use of at least the above solid form of one harringtonine or homoharringtonine described in the invention for preparing the above pharmaceutical composition as (i) chemotherapeutic agent, (ii) enhancer of other chemotherapeutic agents (iii) for inhibiting tumors growth, (iv) for inhibiting mammalian parasites, (v) as immunosuppressive agent, or (vi) as reversal agent.

The present invention further describes a method for treating mammalian tumors which comprises administering to a mammal an antitumor effective amount of the solid form of at least one harringtonine or homoharringtonine described in this invention, by parenteral, topic, subcutaneous or anal mode.

A preferred embodiment of the invention describes a method for treating mammalian tumors which comprises oral administering to a mammal an antitumor effective amount of the solid form of at least one harringtonine or homoharringtonine described in this invention. A further preferred embodiment of the invention describes a method for treating mammalian tumors which comprises implantable pharmaceutical preparation administering to a mammal an antitumor effective amount of the solid form of at least one harringtonine or homoharringtonine described in this invention.

Finally, the invention is also concerned with the use of purified and/or solid harringtonines as defined above, for the preparation of pharmaceutical compositions for the treatment of cancers and leukemias particularly acute myelod leukemia (AML), myelodysplastic syndrome (MDS) and myeloproliferative disorders including chronic myelogenous leukemia.

### EXAMPLE 1

### Preparation of Harringtonine Drug Substance by Purification of Commercial Natural Harringtonine

A. Analytical Profile of Starting Product

By combination of HPLC analysis with UV detection (see FIG. 6) and mass spectrometry detection (see FIG. 7 and 8) a total of 6.5% of related compound (identified as b,c: position isomer of harringtonine=3.4%; d: homoharringtonine=3%; e: 4′-demethyl harringtonine=0.01%; f: drupacine derivative: 0.05%) are found in the starting product.

B. Chromatography of Natural Harringtonine

Natural harringtonine (5 grams) is injected on a preparative high-pressure liquid chromatography (HPLC) system (Prochrom stainless steel; permanent axial compression; diameter: 80 mm; length: 1000 mm) containing 1000 grams of reverse phase octadecylsilane specially dedicated for basic compounds as stationary phase. Then elution is performed in using a gradient of pH 3 buffered methanol-water solution as mobile phase (pressure 1200 psi). Unwanted fractions are discarded based upon in-line UV spectrophotometric detection. Kept fractions are collected in 16 separate containers which each are individually checked in using an analytical HPLC system exhibiting a different selectivity pattern (octadecylsilane as stationary phase and buffered acetonitrile-water system as mobile phase). During the development phase, a dual in-line UV-MS detection is used. After discarding of the fractions representing more than 0.5% of the total content of harringtonine, fractions which complied with pre-established specification were gathered, neutralized then evaporated under reduce pressure. Then crude concentrated solution of harringtonine are alkalinized at pH 8.5 with aqueous ammonia and partitioned with dichloromethane. Resulting organic solution is concentrated under high vacuum. In-process HPLC analysis indicated a total of related compound lower than 1.5%.

C. Crystallization of Raw Harringtonine

Under a laminar flow hood, the above raw harringtonine (4.1 grams) is dissolved in methanol (5 ml), at 30° C. The resulting alcoholic solution was filtered on a 0.25μ sterile Millipore filter to remove microparticules and germs and collected in a sterilized rotary flask. Then, desionized water (50 mL) is added and methanol is completely removed under vacuum at 30° C. in using a decontaminated rotary evaporator. After removing methanol, heating is stopped and the aqueous solution of harringtonine is kept under vacuum and rotation is continued during appearance of white crystals of pure harringtonine. The stirring is continued until no more crystal occurs. Under a laminar flow hood, the suspension of is poured on a sintered glass filter with house vacuum. The resulting crystalline solid cake is washed two times with cold desionized water (10 mL×2). The white translucent crystals are then dried using high vacuum at 40° C. for 24 hours. Overall yield is 76%. All operations were documented prior to start the process and full current Good Manufacturing Practices were applied. This clinical batch corresponds to 400 therapeutic units dosed at 10 mg.

D. Analysis

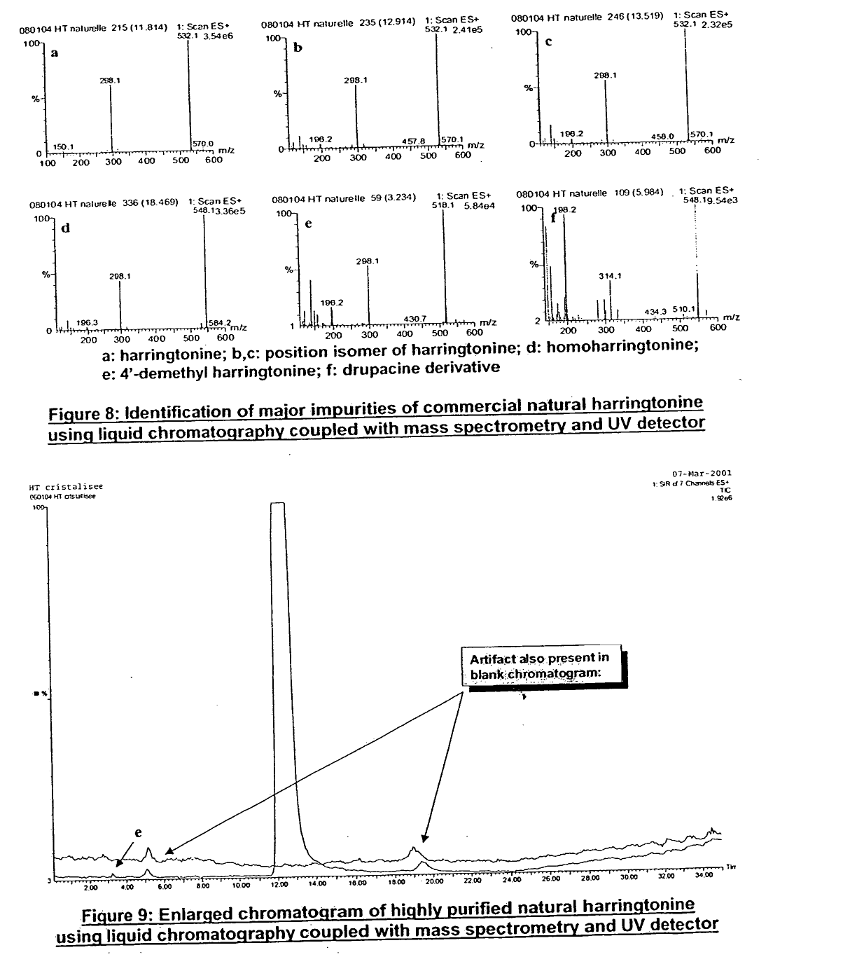

Routine analytical procedure includes solvent residues, loss on drying, water determination, melting point, IR and NMR spectrum, related compound and assay by HPLC. FIG. 7 and 9 compare HPLC chromatogram before and after purification in using this process. Table II shows the comparison of the corresponding related compound content.

TABLE II
Impurity Content Decrease After Application Of This Process

| | | Before | After |
|---|---|---|---|
| Peak | Related Compound (impurities) | this process | this process |
| a | Harringtonine (HA) | 93.49 | 99.97 |
| b | HA isomer | 1.76 | 0 |
| c | HA isomer | 1.67 | 0 |
| d | Homoharringtonine | 3.01 | 0 |
| e | 4′-dmethyl-HA | 0.01 | 0.03 |
| f | Drupacine analog | 0.05 | 0 |
| | Sum of Related Compounds | 6.49 | 0.03 |

| | |
|---|---|
| Rate 6.49/0.03 | 216 |

For the aim of further characterization, more advanced studies were performed including differential scanning calorimetry (DSC) thermogravimetry, 2D NMR, solid NMR and X-ray powder diffractometry.

Infrared Spectrometry:

Identical IR spectra were obtained by either the KBr pellet and/or mineral oil mull preparation technique. FIG. 5 shows typical infrared spectrum (KBr) for unambiguous identification at the solid state of the crystalline harringtonine obtained by this process. A series of sharp absorption bands are noted at 615, 654, 674, 689, 709, 722, 750, 761, 805, 850, 928, 989, 1022, 1033, 1062, 1083, 1112, 1162, 1205, 1224, 1262, 1277, 1308, 1340, 1364, 1382, 1438 1486, 1508, 1625, 1656, 1725, 1745, 2883, 2936, 2972, 3079, 3353, 3552 and 3647 cm⁻¹

Differential Scanning Calorimetry (DSC) And Thermogravimetry (TG)

Measurement of DSC and TG were obtained on a Mettler Toledo STAR System. Approximately 12 mg of harringtonine drug substance were accurately weighed (12.4471 mg) into a DSC pan. The sample was heated from 25° C. to 200° C. at a rate of 10° C./min. The DSC data were obtained following a standard method in the art. The DSC curve of crystalline harringtonine drug substance ((FIG. 4), exhibits a melting endotherm at 79.5° C. No subsequent decomposition occurred under the upper tested temperature 200° C. Simultaneous TG measurement, indicated a loss on drying of 1.3% which did not correspond to a lost of structural molecule of solvent or water.

### EXAMPLE 2

### Preparation of Homoharringtonine Drug Substance by Purification of Raw Semi-synthetic (Hemi-synthetic) Homoharringtonine

A. Analytical Profile of Starting Product

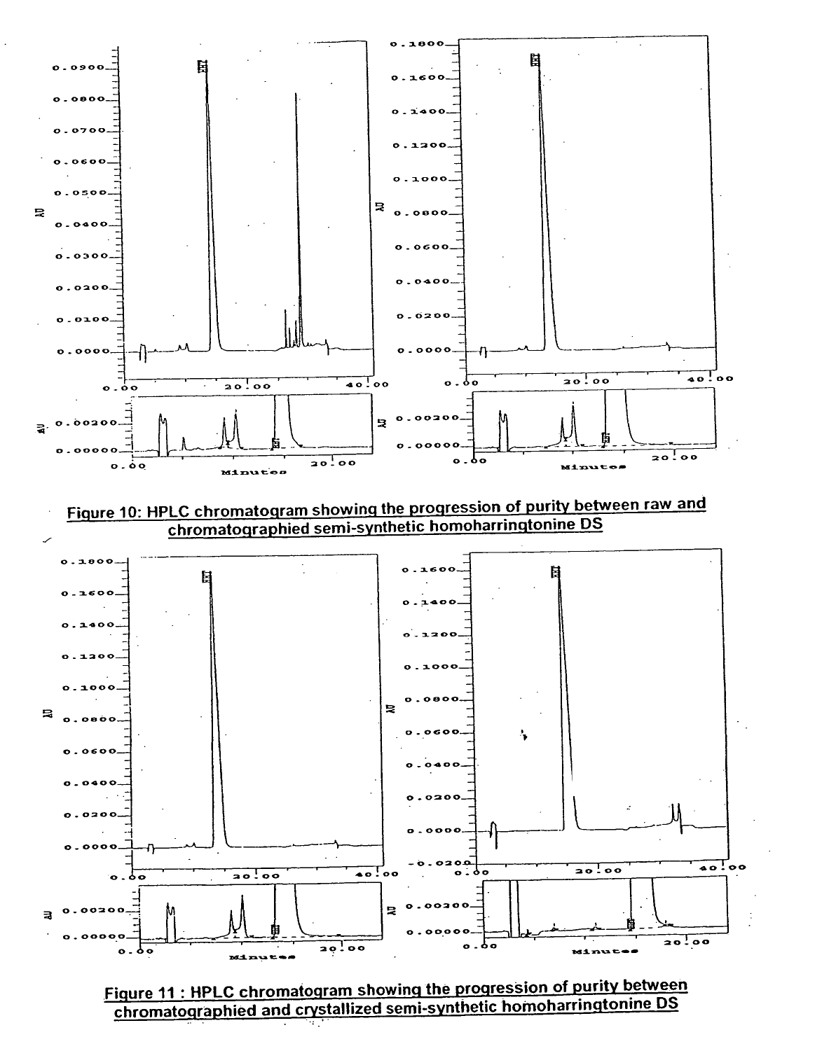

Crude reaction mixture of raw homoharringtonine contains a potential of 250 grams of homoharringtonine DS together with process impurities such as catalyst, unchanged starting product (anhydro-homo-harringtonine), and some related side product. HPLC analysis with UV detection (see left-side chromatogram on FIG. 10) indicated a total of 9% of related impurities.

B. Chromatography of Semi-synthetic Homoharringtonine

Raw semi-synthetic homoharringtonine (550 grams) is injected on a preparative high-pressure liquid chromatography (HPLC) system (Prochrom stainless steel; permanent axial compression; diameter: 450 mm; length: 1000 mm) containing 48,000 grams of reverse phase octadecylsilane specially dedicated for basic compounds as stationary phase. Then elution is performed in using a gradient of pH 3 buffered methanol-water solution as mobile phase (pressure 1200 psi, flow-rate 540 L/hour). Unwanted fractions are discarded based upon by-passed in-line UV spectrophotometric detector. Kept fractions are collected in 30 separate stainless steel containers (20 or 50 L each) which are individually checked in using an analytical HPLC system exhibiting a different selectivity pattern (octadecylsilane as stationary phase and buffered acetonitrile-water system as mobile phase) and equipped with a diode array detector. After discarding of the fractions representing more than 0.5% of the total content of homoharringtonine, fractions which complied with pre-established specification were gathered, neutralized then evaporated under reduce pressure in using a mechanically stirred thin film evaporator. Then crude concentrated solution of homoharringtonine are alkalinized at pH 8.5 with aqueous ammonia and partitioned with dichloromethane. Resulting organic solution is concentrated under high vacuum. In-process HPLC analysis indicated a total of related compound lower than 0.5% (see right-side chromatogram on FIG. 10)

C. Crystallization of Homoharringtonine DS

In a controlled clean room, under a laminar flow hood, the above raw homoharringtonine DS (210 grams) is dissolved in methanol (240 mL), at 30° C. The resulting alcoholic solution is filtered on a 0.25μ sterile Millipore filter to remove microparticules and germs and collected in a sterilized pilot rotary flask. Then, desionized water (2400 mL) is added and methanol is completely removed under vacuum at 30° C. in using a decontaminated pilot rotary evaporator. After removing methanol, heating is stopped and the aqueous solution of homoharringtonine DS is kept under vacuum and rotation is continued during appearance of white crystals of pure homoharringtonine. The stirring is continued until no more crystal occurs. Under a laminar flow hood, the suspension of is poured on a sintered glass filter with house vacuum. The resulting crystalline solid cake is washed two times with cold desionized water (450 mL×2). The white crystals are then dried using high vacuum at 60° C. for 48 hours. Overall yield is 88% from potential content of homoharringtonine in raw semi-synthetic homoharringtonine. All operations were documented prior to start the process and full current Good Manufacturing Practices were applied. This clinical batch corresponds to 40,000 therapeutic units dosed at 5 mg.

D. Analysis

Routine analytical procedure includes solvent residues, loss on drying, water determination, melting point, IR and NMR spectrum, related compound and assay by HPLC. FIG. 11 shows HPLC chromatogram before and after crystallization. Total of related impurities of homoharringtonine DS is 0.03%. For the aim of further characterization, more advanced studies were performed including differential scanning calorimetry (DSC), thermogravimetry (TD), 2D NMR, solid NMR and X-ray powder diffractometry.

Infrared Spectrometry:

Identical IR spectra were obtained by either the KBr pellet and/or mineral oil mull preparation technique. FIG. 3 shows typical infrared spectrum (KBr) for unambiguous identification at the solid state of the crystalline homoharringtonine obtained by this process. A series of sharp absorption bands are noted at 612, 703, 771, 804, 826, 855, 879, 932, 1029, 1082, 1119, 1135, 1161, 1191, 1229, 1274, 1344, 1367, 1436, 1457, 1488, 1505, 1653, 1743, 2814, 2911, 2958, 3420, and 3552 cm⁻¹

Differential Scanning Calorimetry (DSC) And Thermogravimetry (TG)

Measurement of DSC and TG were obtained on a Mettler Toledo STAR System. Approximately 11 mg of homoharringtonine drug substance were accurately weighed (10.6251 mg) into a DSC pan. The sample was heated from 25° C. to 250° C. at a rate of 5° C./min. The DSC data were obtained following a standard method in the art. The DSC curve of crystalline homoharringtonine drug substance (FIG. 1), exhibits a melting endotherm at 145.6° C. Melting range performed by the capillary method (Bucchi Apparatus) gave 143-145° C. Literature indicated 144-146° C. [Anonymous, Acta Bot. Sin. 22, 156 (1980) cited by L. Huang and Z. Xue, Cephalotaxus Alkaloids, in “The Alkaloids”, vol. XXIII, pp157, (1988). Crystallization medium was not published. This is the only literature reference regarding melting point of a crystalline form of HHT]

X-Ray Powder Diffraction

X-ray powder diffraction pattern was collected on a INEL microdiffractomer, model DIFFRACTINEL. Powdered homoharringtonine DS was packed in a glass capillary tube and was analyzed according to a standard method in the art. The X-ray generator was opered at 45 kV and 40 mA, using the copper Kalpha line as the radiation source. The sample was rotated along the chi axis and data was collected between 0 and 120 deg 2-theta. A collection time of 1200 sec was used. As showed on FIG. 2, the x-ray powder diffraction for this crystalline form of homoharringtonine shows a typical pattern including major reflection peaks at approximately 7.9, 9.2, 10.9, 14.9 16.0, 17.7, 19.5, 19.7, 21.78, 23.1, 25.3, 25.4 and 25.7 deg 2-theta.

### EXAMPLE 3

### Preparation of Homoharringtonine Drug Substance by Purification of a Commercial Sample of Impure Homoharringtonine from Chinese Source

A. Analytical Profile of Starting Product

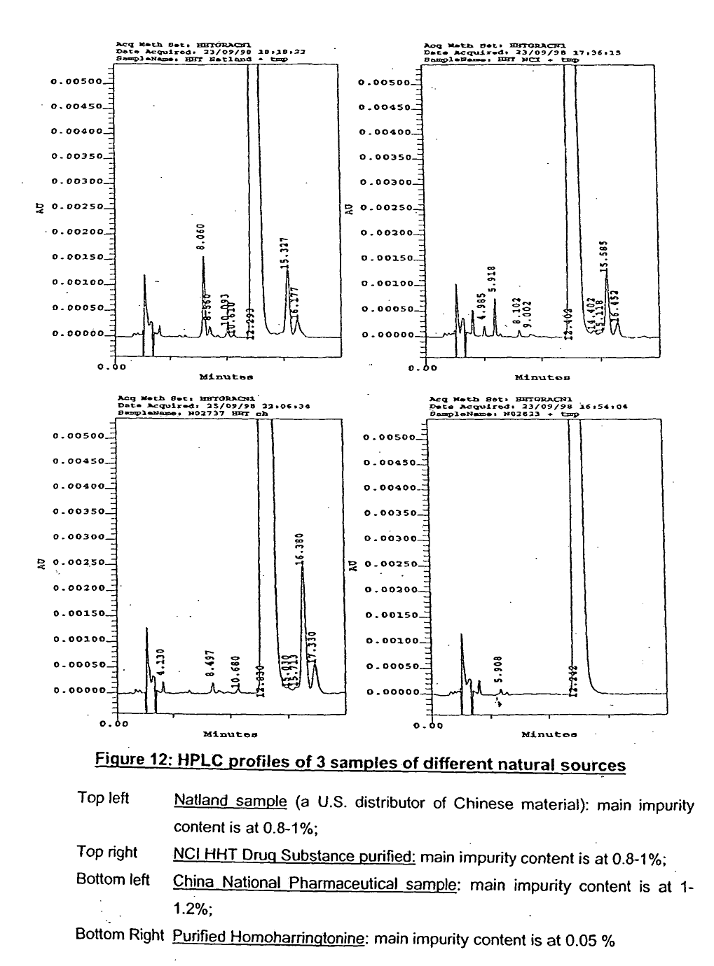

Analytical HPLC chromatogram of natural homoharringtonine (China National Pharmaceutical) is displayed on FIG. 12 (bottom left).

B. Chromatography of Natural Homoharringtonine

Natural homoharringtonine (25 grams) is injected on a preparative high-pressure liquid chromatography (HPLC) system (Prochrom stainless steel; permanent axial compression; diameter: 200 mm; length: 1000 mm) containing 12,000 grams of reverse phase octadecylsilane specially dedicated for basic compounds as stationary phase. Then elution is performed in using a gradient of pH 3 buffered methanol-water solution as mobile phase (pressure 1200 psi, flow-rate 120 L/hour). Unwanted fractions are discarded based upon by-passed in-line UV spectrophotometric detector. Kept fractions are collected in 22 separate stainless steel containers which are individually checked in using an analytical HPLC system exhibiting a different selectivity pattern (octadecylsilane as stationary phase and buffered acetonitrile-water system as mobile phase) and equipped with a diode array detector. After discarding of the fractions representing more than 0.5% of the total content of homoharringtonine, fractions which complied with preestablished specification were gathered, neutralized then evaporated under reduce pressure in using a mechanically stirred thin film evaporator. Then crude concentrated solution of homoharringtonine are alkalinized at pH 8.5 with aqueous ammonia and partitioned with dichloromethane. Resulting organic solution is concentrated under high vacuum. In-process HPLC analysis indicated a total of related compound lower than 0.5%.

C. Crystallization of Homoharringtonine DS

In a controlled clean room, under a laminar flow hood, the above chromatographied homoharringtonine DS (18 grams) is dissolved in methanol (35 mL), at 30° C. The resulting alcoholic solution is filtered on a 0.25μ sterile Millipore filter to remove microparticules and germs and collected in a sterilized pilot rotary flask. Then, desionized water (300 mL) is added and methanol is completely removed under vacuum at 30° C. in using a decontaminated pilot rotary evaporator. After removing methanol, heating is stopped and the aqueous solution of homoharringtonine DS is kept under vacuum and rotation is continued during appearance of white crystals of pure homoharringtonine. The stirring is continued until no more crystal occurs. Under a laminar flow hood, the suspension of is poured on a sintered glass filter with house vacuum. The resulting crystalline solid cake is washed two times with cold desionized water (50 mL×2). The white crystals are then dried using high vacuum at 60° C. for 48 hours. Overall yield is 84% from potential content of homoharringtonine in raw semi-synthetic homoharringtonine. All operations were documented prior to start the process and full current Good Manufacturing Practices were applied.

D. Analysis

Routine analytical procedure includes solvent residues, loss on drying, water determination, melting point, IR and NMR spectrum, related compound and assay by HPLC. FIG. 12 (bottom right) shows HPLC chromatogram after crystallization. Total of related impurities of homoharringtonine DS is 0.05%.

For the aim of further characterization, more advanced studies were performed including differential scanning calorimetry (DSC), thermogravimetry (TD), 2D NMR, solid NMR and X-ray powder diffractometry.

Infrared Spectra, Differential Scanning Calorimetry (DSC) and X-Ray Powder Diffraction gave patterns strictly superimposable to the one of example 2 obtained from semi-synthetic homoharringtonine (FIG. 3, 1, and 2, respectively).

1. Natural, synthetic or semi-synthetic harringtonines including their tautomeric forms and their salts of the following formula: 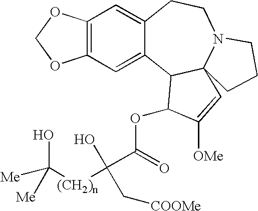
wherein n=2 (i.e. harringtonine) or n=3 (i.e. homoharringtonine), in which
the total content of impurities, possibly including enantiomeric forms, is lower than 1%, and/or
the content of the major impurity is lower than 0.9%, and/or
the chromatographic assay exhibits a harringtonines content higher than 97.5%.

2. Natural, synthetic or semi-synthetic homoharringtonine including its tautomeric forms and its salts in which:
the total content of impurities, possibly including enantiomeric forms, is lower than 1%, and/or
the content of the major impurity is lower than 0.9%, and/or
the chromatographic assay exhibits a homoharringtonine content higher than 97.5%.

3. Natural synthetic or semi-synthetic harringtonine including its tautomeric forms and its salts in which:
the total content of impurities, possibly including enantiomeric forms, is lower than 1%, and/or
the content of the major impurity is lower than 0.9%, and/or
the chromatographic assay exhibits a harringtonine content higher than 97.5%.

4. A crystalline natural, synthetic or semi-synthetic homoharringtonine having substantially the same DSC curve as set out in FIG. 1.

5. A crystalline natural, synthetic or semi-synthetic homoharringtonine having substantially the same X-ray diffractogram as set out in FIG. 2, and substantially the same IR spectrum, in KBr as set out in FIG. 3.

6. A crystalline natural, synthetic or semi-synthetic homoharringtonine having substantially the same DSC curve as set out in FIG. 1, and substantially the same X-ray diffractogram as set out in FIG. 2, and substantially the same IR spectrum, in KBr as set out in FIG. 3.

7. A crystalline natural, synthetic or semi-synthetic harringtonine having substantially the same DSC curve as set out in FIG. 4

8. A crystalline natural, synthetic or semi-synthetic harringtonine having substantially the same IR spectrum, in KBr as set out in FIG. 5.

9. A crystalline natural, synthetic or semi-synthetic harringtonine having substantially the same DSC curve as set out in FIG. 4, and substantially the same IR spectrum, in KBr as set out in FIG. 5

10. A pharmaceutical composition comprising an effective antitumor amount of one or more natural, synthetic or semi-synthetic harringtonines having each a total of impurities content lower than 1% or exhibiting its major impurity at a level of content lower than 0.9%, alone or in combination with one or more other active components, together with one or more other pharmaceutically acceptable inactive components such as carriers, excipients, adjuvants or diluents.

11. A pharmaceutical composition comprising an effective antitumor amount of a natural, synthetic or semi-synthetic homoharringtonine having a total of impurities content lower than 1% or exhibiting its major impurity at a level of content lower than 0.9%, together with one or more pharmaceutically acceptable inactive components such as carriers, excipients, adjuvants or diluents.

12. A pharmaceutical composition comprising an effective antitumor amount of a natural, synthetic or semi-synthetic harringtonine having a total of impurities content lower than 1% or exhibiting its major impurity at a level of content lower than 0.9%, together with one or more pharmaceutically acceptable inactive components such as carriers, excipients, adjuvants or diluents.

13. A pharmaceutical composition comprising an effective antitumor amount of a natural, synthetic or semi-synthetic homoharringtonine having substantially the same X-ray diffractogram as set out in FIG. 2, and substantially the same IR spectrum, in KBr as set out in FIG. 3, together with one or more pharmaceutically acceptable inactive components such as carriers, excipients, adjuvants or diluents.

14. A pharmaceutical composition comprising an effective antitumor amount of a natural, synthetic or semi-synthetic homoharringtonine having substantially the same X-ray diffractogram as set out in FIG. 2, and substantially the same IR spectrum, in KBr as set out in FIG. 3, and substantially the same DSC curve as set out in FIG. 1, together with one or more pharmaceutically acceptable inactive components such as carriers, excipients, adjuvants or diluents.

15. A pharmaceutical composition comprising an effective antitumor amount of a natural, synthetic or semi-synthetic harringtonine having substantially the same IR spectrum, in KBr as set out in FIG. 5, and substantially the same DSC curve as set out in FIG. 4, together with one or more pharmaceutically acceptable inactive components such as carriers, excipients, adjuvants or diluents.

16. A process of purification of natural, synthetic or semi-synthetic crude harringtonines for the preparation of pure harringtonines exhibiting the features included in claims 1 to 9 including for eventual enantiomeric enrichment, and comprising
one or more chromatographic purification(s)
one or more crystallization(s) in which the solvent is water or a lower C₁₋₄ alkanol or an aqueous solvent mixture containing one or more organic solvents

17. The process of claim 16 in which the solvent is water.

18. The process of claim 16 in which the solvent is water or an aqueous solvent mixture containing one or more organic solvents

19. The process of claim 16 in which the organic solvent mixture includes one lower C₁₋₄ alkanol or more.

20. The process of claims 16 and 19 in which the lower alkanol is methanol.

21. The process of purification of claims 16 to 20 in which the chromatographic purification is in reverse phase, the solvent is an aqueous solvent containing a lower C₁₋₄ alkanol, a solvent mixture of equivalent selectivity and, eventually, a buffer, preferably based on phosphoric acid and is salt

22. The process of claims 16 to 21, in which the harringtonine is homoharringtonine

23. The process of claims 16 to 21, in which the harringtonine is harringtonine

24. The use of purified and/or solid harringtonines as defined in claims 1 to 9 for preparing pharmaceutical composition as defined in claims 10 to 15 for treatment of mammal diseases.

25. The use of purified and/or solid harringtonines as defined in claims 1 to 9 for preparing pharmaceutical composition as defined in claims 10 to 15 for treatment of tumors or parasitic disease, or as immunosuppressive therapy or reversal agent.

26. The use of purified and/or solid harringtonines as defined in claims 1 to 9 for preparing pharmaceutical composition as defined in claims 10 to 15 for treatment of cancers and leukemias particularly acute myeloid leukemia (AML), myelodysplastic syndrome (MDS) and, myeloproliferative disorders including chronic myelogenous leukemia, including in combination with other agents.

27. The use of the purified and/or solid harringtonines or their salts as defined in claims 1 to 9 for preparing a pharmaceutical composition as defined in claims 10 to 15 for treatment of parasitic diseases.

28. The use of purified and/or solid harringtonines as defined in claims 1 to 9 for preparing a pharmaceutical composition as defined in claims 10 to 15 as adjuvent therapy of resistance to other chemotherapeutic agents.

29. The method of treatment of claims 24 to 28 in which the drug is given by parenteral mode of administration.

30. The method of treatment of claims 24 to 28 in which the drug is given by oral mode of administration.

31. The method of treatment of claims 24 to 28 in which the drug is given by anal mode of administration.

32. The method of treatment of claims 24 to 28 in which the drug is given by topic mode of administration.

33. The method of treatment of claims 24 to 28 in which the mode of administration of the drug is an implant.

34. The method of treatment of claim 29 in which the parenteral mode of administration is subcutaneous.
